(19) United States
(12) Patent Application Publication
(10) Pub. No.: US 2010/0004531 A1
(43) Pub. Date: Jan. 7, 2010

(54) MEASUREMENT CATHETER

(76) Inventor: Charles G. PASSMORE, La Jolla, CA (US)

Correspondence Address: DALINA LAW GROUP, P.C., 7910 IVANHOE AVE. #325, LA JOLLA, CA, 92037, US

(21) Appl. No.: 12/168,702

(22) Filed: Jul. 7, 2008

(51) Int. Cl. A61B 5/026; A61B 5/05

(52) U.S. Cl. 600/425; 600/505

(57) ABSTRACT

Enables a measurement catheter configured to hold a package such as an optical tomographic module and/or blood flow velocity module. May hold any package reduced to a size small enough to fit in a blood vessel. Example packages include an optical transmitter and receiver/detectors that enable internal optical tomographic images of vessels to be captured, for example without rotation of the catheter in the vessel. Alternatively or in combination, a thermal package may be coupled to the measurement catheter that includes a thermal element and detector(s) that enable blood flow velocity to be accurately internally measured that does not require a static catheter position. In addition, the measurement catheter may optionally attach to an interchangeable coupler that allows for the introduction of substitution of packages or any type of catheter end assembly to provide rapid deployment of additional surgical or sensory elements.

### BACKGROUND OF THE INVENTION

1. Field of the Invention

Embodiments of the invention described herein pertain to the field of medical devices. More particularly, but not by way of limitation, one or more embodiments of the invention enable a measurement catheter configured to hold a package such as an optical tomographic module and/or blood flow velocity module for example with an optional interchangeable coupler near the insertion end of the measurement catheter.

2. Description of the Related Art

Catheters are tubular devices inserted into a blood vessel or other body cavity to permit the extraction or injection of fluids or to allow access to an internal portion of the body for a surgical instrument. Catheters are generally utilized for tasks such as heart related surgeries or obtaining internal images of ones body. Some examples of heart related uses for catheters include angioplasty, angiography and balloon septostomy.

Angioplasty relates to physical widening of obstructed blood vessels. The obstructed blood vessels may be caused by atherosclerosis for example. The use of a catheter to widen an obstructed vessel allows for percutaneous access without requiring a scalpel for example to open the body. By introducing the catheter through the lumen of a needle, disruption of the tissue is thus minimized. Introducing a catheter through the lumen of the needle is known as the “modified Seldinger technique.” Positioning of the catheter is generally handled through the use of Flouroscopy which uses X-rays to enable the surgeon to view the catheters internal position in real time during the procedure. In some cases, surgical removal of fatty plaque buildup within the blood vessels is utilized to improve blood flow. When removing or breaking up these fatty plaques, there is no guarantee that some of the plaque will not break free and cause a problem in another part of the body, for example a stroke.

Angiography is a medical imaging technique wherein X-ray images are taken to visualize the inner portion of blood vessels such as the arteries, veins and chambers of the heart for example. Depending on the type of imaging, the catheter is generally inserted into the jugular vein or femoral artery. Typical imaging utilizes X-rays to be produced and captured at a few frames per second. This allows the cardiologist to view constricted portions of a vessel for example.

Balloon septostomy also relates to widening of inner portions of the body, such as a blood vessel, using a balloon catheter. A balloon catheter includes an inflatable balloon at the tip that is deflated and positioned at the desired location. Once inflated, plaque within the artery is compressed. Alternatively, a stent may be expanded with the balloon and left behind in the artery after the balloon is deflated and removed from the artery.

Current blood flow velocity catheter devices utilize Doppler crystals to measure blood flow velocity. The measurement of blood flow velocity requires a static position of the catheter so that the indirect reflections of various arterial structures remains constant while the frequency shift of the return acoustic signal is measured. Current optical coherent tomographic catheters rely on rotation of the catheter with complex motor and connector assemblies that have many mechanical parts with limited lifetimes. For at least these reasons, there is a need for a measurement catheter as enabled herein.

### BRIEF SUMMARY OF THE INVENTION

One or more embodiments of the invention enable a measurement catheter configured to hold a package such as an optical tomographic module and/or blood flow velocity module. Embodiments of the invention may hold any package reduced to a size small enough to fit in a blood vessel. Some example of the type of packages that are appropriate for use within a blood vessel include packages such as an optical transmitter and receiver/detectors that enable internal optical tomographic images of vessels to be captured, for example without rotation of the catheter in the vessel. Alternatively or in combination, a thermal package may be coupled to the measurement catheter that includes a thermal element and detector(s) for enabling internal blood flow velocity to be accurately measured in a way that does not require a static catheter position. Other examples of packages for use within blood vessels may include chemical sensors or oxygen sensors, or ultrasound packages. In addition, the measurement catheter may optionally attach to an interchangeable coupler that enables the introduction of substitution of packages or any type of catheter end assembly to provide rapid deployment of additional surgical or sensory elements.

In one or more embodiments of the invention blood flow velocity may be measured inside a blood vessel near a stenosis which is an abnormal narrowing of the blood vessel. The stenosis may be removed from the inside of a blood vessel via any type of catheter extension configured to remove matter from the inner portion of a blood vessel. The blood flow velocity may be measured again or during the surgical procedure and if the blood flow velocity reaches a desired rate, the procedure may thus end.

One or more embodiments of the blood flow velocity package function by introducing a temperature change to the blood at a first position on the package that is measured at a second position on the package by a thermal detector. The distance between the first and second position is utilized with the measured time offset from the introduction of the temperature to determine the velocity, i.e., the velocity is equal to the distance between the positions divided by the measured time offset. Since one or more embodiments of the invention provide a blood flow velocity package configured to calculate the velocity of insertion of the catheter, the device may also obtain blood flow velocity during insertion or extraction. This capability is present in the device without requiring a static catheter position. For example, subtracting the insertion velocity from the measured blood flow velocity enables the determination of blood flow measurements during insertion, e.g., against the direction of blood flow. Furthermore, adding the insertion velocity to the measured blood flow velocity allows for blood flow measurements during insertion, e.g., in the direction of blood flow. Likewise, blood flow velocity may also be calculated during extraction of the catheter by adding the extraction velocity if moving the catheter in the direction of blood flow, or subtracting the extraction velocity if moving the catheter in the opposite direction of blood flow.

Alternatively or in combination, an optical tomographic package coupled to the measurement catheter may be utilized to obtain an internal optical tomographic image of the vessel at or near the stenosis to determine the shape, depth or other physical features of the stenosis. The capturing of internal optical tomographic images may be performed before, during and/or after a surgical procedure for example to determine the effects of the procedure.

One or more embodiments of the tomographic package may direct light radially away from the package with fiber optic line(s) and receive light back at any number of positions around the radius of the package without rotating the package. The more positions utilized to receive light, the higher the resolution of the resulting image. There are many methods for receiving light around the radius of the package including multiple fibers spaced about the package and pointing away from the package so as to receive light that has been transmitted by one or more outwardly pointing transmitting fibers. Embodiments of the invention thus do not require rotation of the catheter to obtain images around the axis of the catheter. Referring specifically now to embodiments of the measurement catheter itself the catheter comprises a thermal element such as a heating or cooling element coupled with the catheter wherein the thermal element is configured to alter blood temperature as the blood flows through a vessel. A thermal sensor or plurality of thermal sensors is coupled with the catheter at an offset from the thermal element. The thermal sensor is configured to measure the blood temperature and blood flow velocity is calculated as the offset divided by a time difference from a point in time when the blood temperature is altered at the thermal element to a point in time wherein an altered blood temperature is measured at the thermal sensor. In one embodiment of the invention an optical transmitter (e.g., a broadband light source, diode, superluminescent diode) and optical sensor is coupled with the catheter. The optical sensor receives coherent radiation transmitted from the optical transmitter to generate an optical tomographic image of the vessel without rotation of the catheter. The measurement catheter may comprise an interchangeable coupler configured to mechanically couple a package to said measurement catheter.

In one or more embodiments of the invention the measurement catheter comprises a thermal element (e.g., a heating or cooling unit) coupled with the catheter wherein the thermal element is configured to alter blood temperature as the blood flows through a vessel. The catheter also contains a thermal sensor or a plurality of thermals sensors coupled with the catheter at an offset from the thermal element wherein the thermal sensor is configured to measure the blood temperature and blood flow velocity is calculated as the offset divided by a time difference from a point in time when the blood temperature is altered at the thermal element to a point in time wherein an altered blood temperature is measured at the thermal sensor. The measurement catheter may comprise an interchangeable coupler configured to mechanically couple a package to said measurement catheter.

In another embodiment of the invention the measurement catheter comprises an optical transmitter (e.g., a broadband light source, diode, superluminescent diode) coupled with the catheter and an optical sensor coupled with the catheter wherein the optical sensor receives coherent radiation transmitted from the optical transmitter to generate an optical tomographic image of the vessel without rotation of the catheter.

### BRIEF DESCRIPTION OF THE DRAWINGS

The above and other aspects, features and advantages of the invention will be more apparent from the following more particular description thereof, presented in conjunction with the following drawings wherein:

FIG. 1 is a view of the measurement catheter having a package coupled to a catheter optionally via an interchangeable coupler, along with a perspective view of the measurement catheter inside a blood vessel that is an exploded view of the blood vessel in the leg of a patient.

FIG. 2 is a close up of the measurement catheter in a blood vessel.

FIG. 3 is a reconstructed three-dimensional view of a blood vessel as captured via an optical tomographic package coupled with the measurement catheter.

FIG. 4 is a reconstructed three-dimensional cutaway view of the blood vessel as captured via an optical tomographic package coupled with the measurement catheter.

FIG. 5 is a view of an embodiment of a catheter measurement device configured to measure the distance, velocity or any other derivative of distance with respect to time of the measurement catheter inside a patient.

FIG. 6 is a view of an optical tomographic package having pairs of fiber optic output and input strands pointing radially outward from the package and configured to provide and gather light for optical tomography.

FIG. 7 shows views of an electro-optical and a mechanical embodiment of the radial transmitting and receiving portion of the fiber optic elements that direct light to/from the optical tomographic element.

FIG. 8 is a view of a thermal package having a thermal element and thermal sensor configured to measure blood flow velocity.

FIG. 9 is a time display of the thermal sensor after introduction of a thermal change in the blood temperature at the thermal element to determine blood flow velocity.

FIG. 10 is a view of the measurement catheter coupled with a tool for use inside of a blood vessel for example.

FIG. 11 is a flowchart showing the steps for manufacturing an embodiment of the measurement catheter that includes thermistors for use as a thermal element and thermal sensor(s).

FIG. 12 is a picture of a thermal embodiment of the measurement catheter.

FIG. 13 is a photograph of the thermistor for use in the thermal embodiment as shown on a human finger.

FIG. 14 is a picture of a second thermal embodiment of the measurement catheter.

FIG. 15 is a close-up picture of the thermal sensor portion of the thermal embodiment of the measurement catheter.

FIG. 16 is a close-up picture of the thermal element portion of the thermal embodiment of the measurement catheter.

### DETAILED DESCRIPTION OF THE INVENTION

A measurement catheter will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the invention.

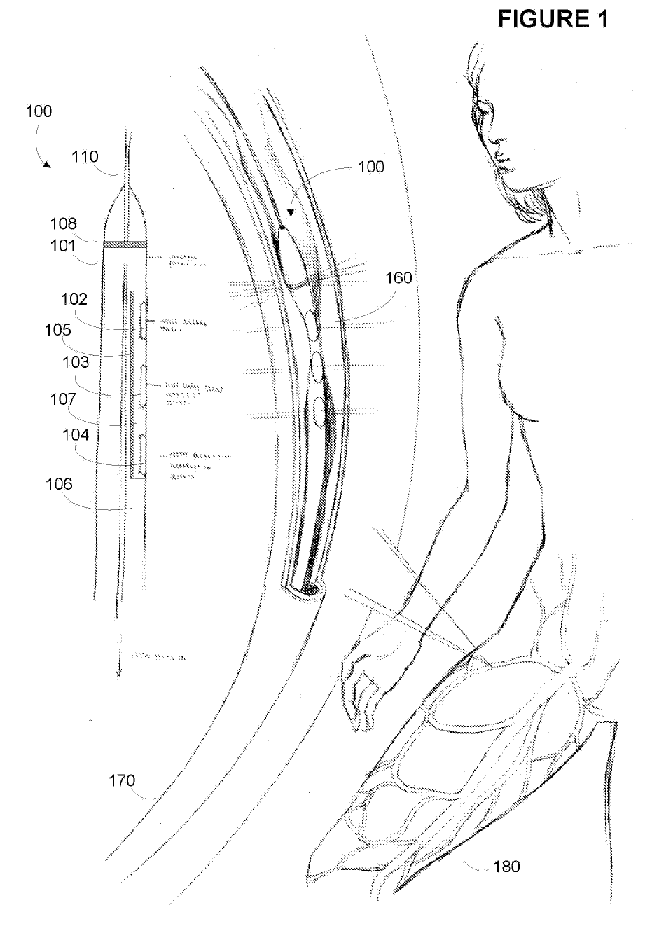

FIG. 1 illustrates a view of measurement catheter 100 having package 107 coupled to catheter 106 via optional interchangeable coupler 105. The “French catheter scale” is utilized in measuring the outer diameter of catheter 100. The diameter of the catheter in millimeters is determined by dividing the “French size” by 3. For example, if the French size is 12, then the diameter in millimeters is 4. The French catheter scale is generally abbreviated “Fr”. Measurement catheter 100 may be of any desired Fr size.

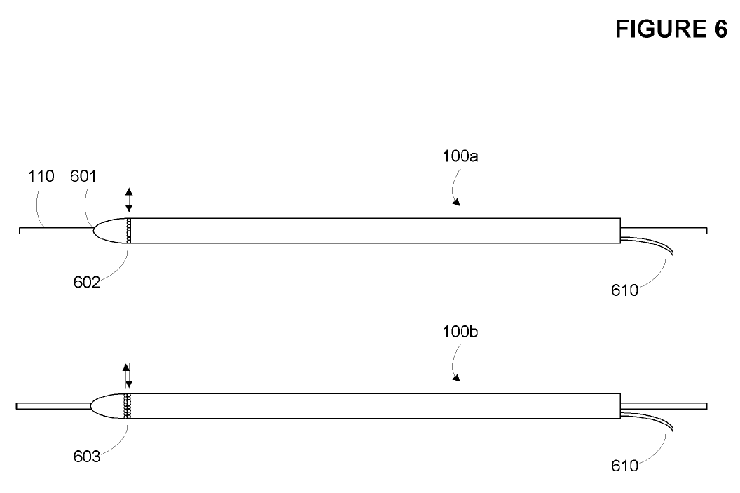

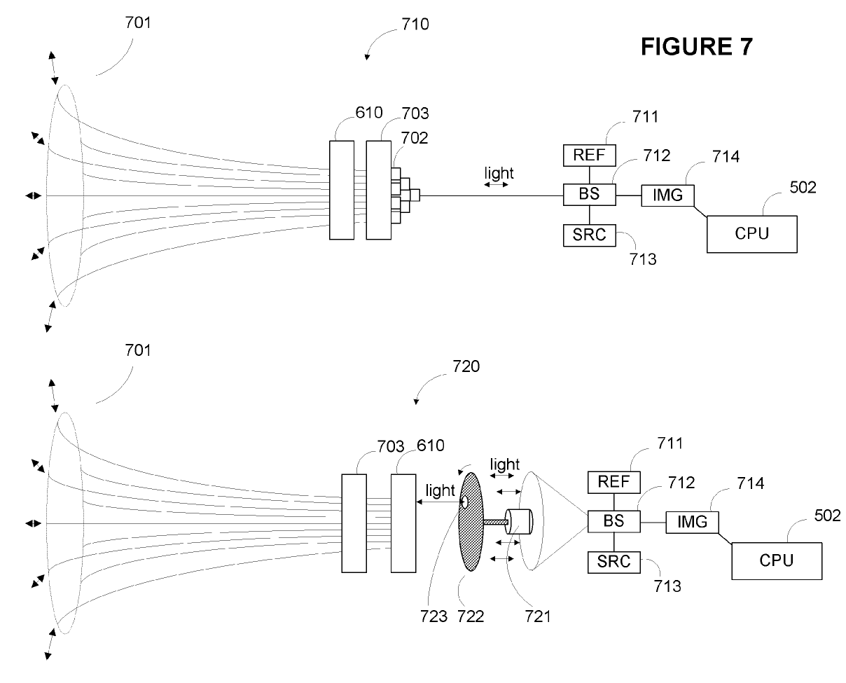

Also shown in FIG. 1 on the right side of the page is a perspective view of measurement catheter 100 inside blood vessel 170 that is an exploded view of blood vessel 170 in the leg 180 of a patient. Measurement catheter 100 as shown includes a optical tomographic element 101, embodiments of which are implemented with single or paired fiber optic elements pointing radially outward from measurement catheter 100 to provide and gather light for optical tomography at an angle away from up to orthogonally away from the axis of guidewire 110 without requiring rotation of the catheter. (See FIGS. 6 and 7) In addition, the embodiment of the invention shown in FIG. 1 includes thermal element 102 and thermal sensors 103 and 104 at various distances away from thermal element 102 along the axis of guidewire 110. Although the thermal package may include one thermal element and one thermal sensor or any number of elements and sensors, this example shows an embodiment with one element and two sensors. The sensors may be utilized to obtain various coarse or fine-grained sensor data and may be averaged to obtain a resulting blood flow velocity for example. When thermal element 102 introduces a temperature change to the blood inside of blood vessel 170 for example, the resulting change in temperature in the blood is detected at thermal sensor 103 after a particular time delay that is dependent on the blood flow velocity in blood vessel 170. The thermal elements and sensors may in one or more embodiments of the invention be implemented with thermistors that are resistors that change resistance based on temperature. Metal ring 108 may be utilized to position measurement catheter 100 inside a body via X-rays for example.

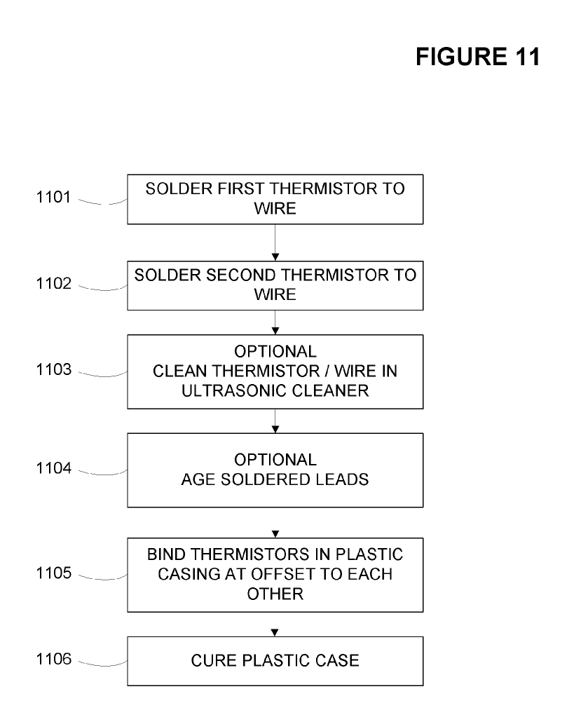

In addition, surgical, balloon or stent elements may be coupled to the measurement catheter to allow for example balloon septostomy and/or stent placement while obtaining simultaneous optical tomographic images and blood velocity flow rates. (See FIG. 11 for example). Alternatively, the measurement catheter may utilize the optical tomographic elements or thermal elements independently with various surgical instruments if desired. For example a thermal blood flow velocity embodiments coupled with a plaque removal device may be utilized to instantaneously determine when enough of the plaque has been removed to restore sufficient blood flow.

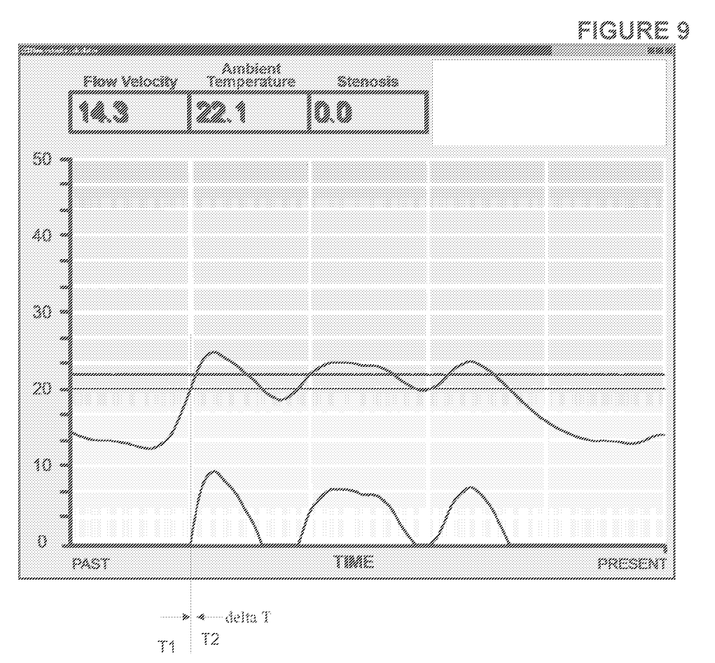

For the blood flow velocity embodiments of the invention, blood flow velocity is determined by measuring the time delay between introducing a thermal change to the blood at thermal element 102 and observing the thermal change at thermal sensor 103 and dividing the distance between thermal element 102 and thermal sensor 103 by the time delay. (See FIG. 9). By also measuring the time delay of the temperature change at thermal sensor 104, and dividing by the distance between thermal element 102 and thermal sensor 103, the first velocity calculation can be verified or averaged for example. As with thermal sensor 103, the next thermal sensor may also be utilized to verify the first two measurements of flow velocity or may be averaged to produce an average result of blood flow. As thermal elements and thermal sensors have varying time delays in introducing or detecting thermal events, these time delays may be subtracted from the time difference of initiating a thermal event at thermal element 102 and detecting the thermal event at thermal sensors 103 and 104 for example. Measurement catheter may be positioned in blood vessel 170 using catheter tube 106 to push or pull package 107 along guidewire 110. In one embodiment of the invention, a blood flow velocity may be taken before and after angioplasty or balloon septostomy for example to determine the change in blood flow velocity. Alternatively, the middle thermal element may introduce a thermal event to the blood that is observed at one of the outer thermal sensors to account for placement of the measurement catheter that is independent of blood flow direction. By observing the thermal event on one or the other “outside” thermistors, as introduced by the middle thermal element (using thermal sensor 103 as a thermal element for example) the blood can thus be determined to flow in the direction from the middle thermistor to the direction of the thermistor that observed the thermal event.

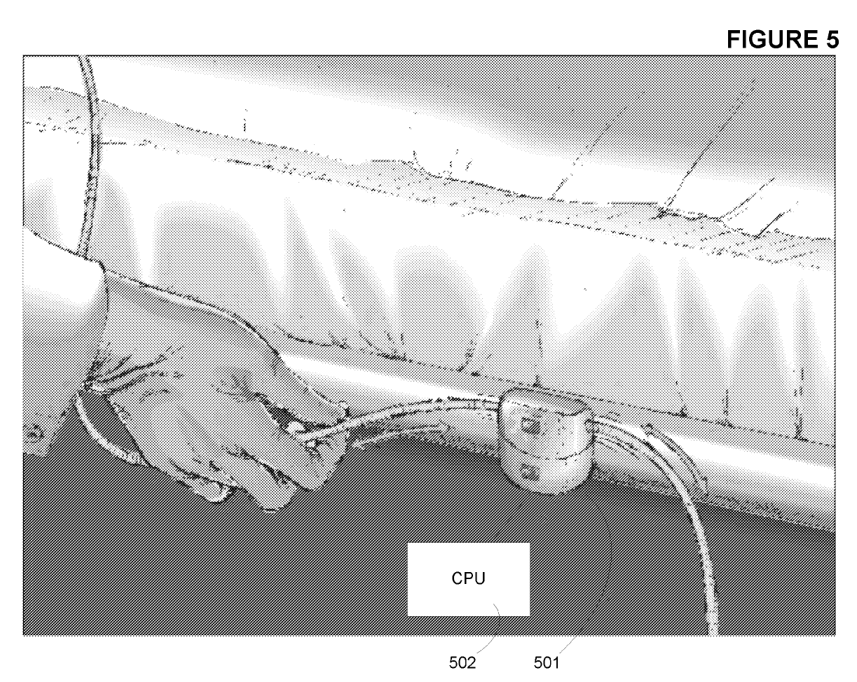

Due to the microscopic size of thermistors, extremely small embodiments of the blood flow velocity catheter embodiments may be constructed by placing two or more thermistors along an axis of the catheter and covering them in medical plastic for example. In addition, since the reflection of sound waves is not utilized as is performed with Doppler catheters, embodiments of the measurement catheter may obtain blood velocity measurements while the measurement catheter is being moved through the vessels. Since embodiments of the invention permit calculation of the velocity of insertion of the catheter, the blood flow velocity may be obtained during insertion or extraction without requiring a static catheter position. For example, subtracting the insertion velocity from the measured blood flow velocity allows for blood flow measurements during insertion, e.g., against the direction of blood flow. Furthermore, adding the insertion velocity to the measured blood flow velocity allows for blood flow measurements during insertion, e.g., in the direction of blood flow. Blood flow velocity may also be calculated during extraction of the catheter by adding the extraction velocity if moving the catheter in the direction of blood flow, or subtracting the extraction velocity if moving the catheter in the opposite direction of blood flow. See FIG. 5 for an embodiment of catheter measurement device 501 that enables the determination of distance and velocity of the catheter with respect to the insertion point. A computer system may be utilized to accept the distance and velocity obtained from catheter measurement device 501 and calculate the true blood flow velocity (after taking into account the direction of blood flow travel, e.g., along or away from the direction of insertion).

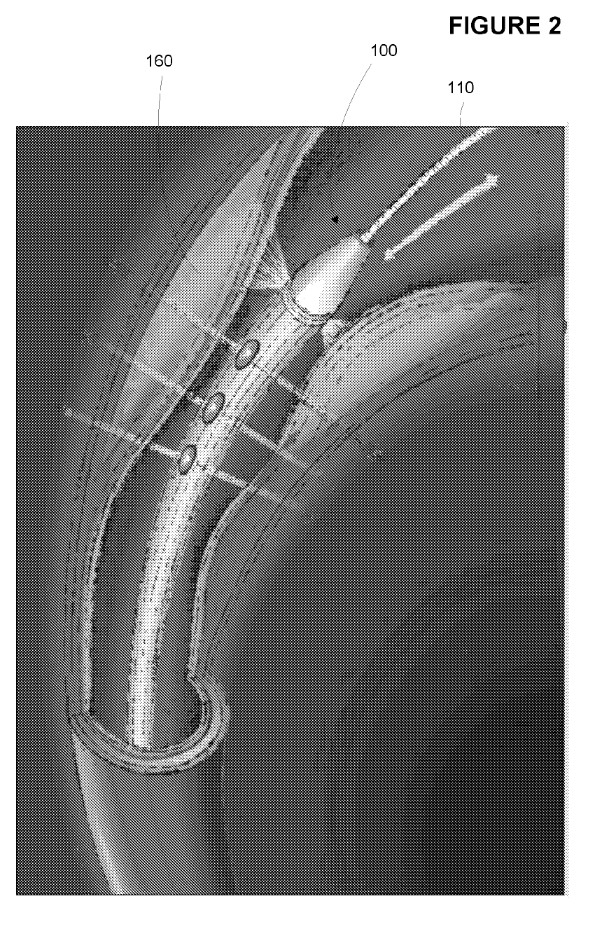

FIG. 2 illustrates a close up of measurement catheter 100 in blood vessel 170. As shown in the figure, the measurement catheter may be moved along guidewire 110 while measuring blood flow and/or performing tomographic image capture. In one or more embodiments of the invention, the measurement catheter may include one measurement device or multiple measurement devices, each of which may or may not be of the same type (as shown). Alternatively, other embodiments of the invention may include a single type of measurement instrument, e.g., a thermal package or a tomographic package that is not combined on one catheter for example. In addition, other types of tools may be combined onto the measurement catheter to allow for internal work to be performed while measuring blood flow or performing tomographic image capture (or ultrasound or any other type of measurement as per the desired package coupled to the measurement catheter).

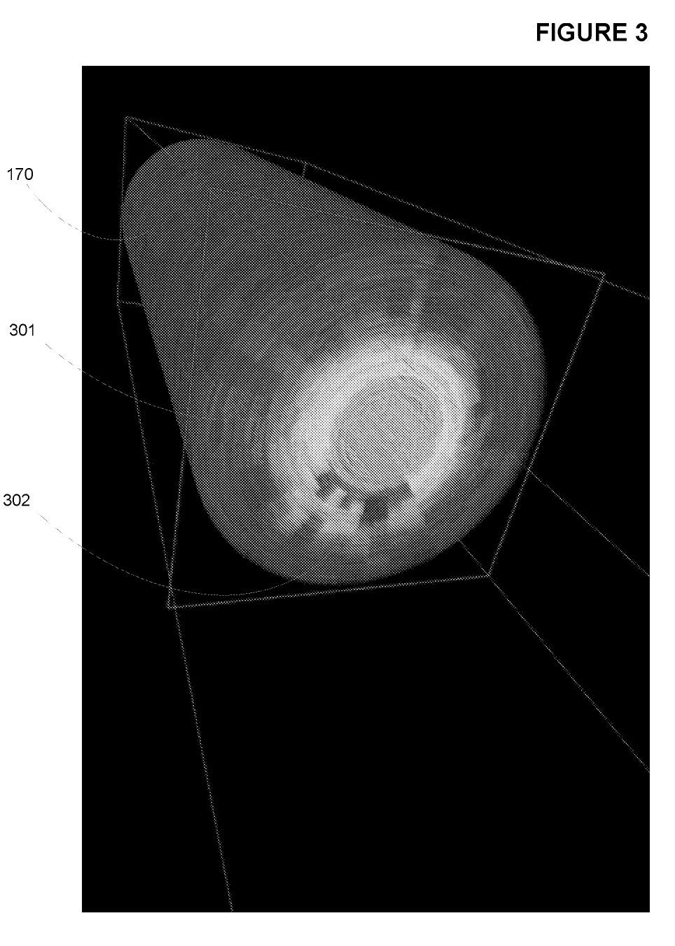

FIG. 3 is a reconstructed three-dimensional view of blood vessel 170 as captured via an optical tomographic package coupled with the measurement catheter. Plaque 301 is shown as constricting blood vessel 170, while lesion or endothelial disorder 302 shows a potential problem as the blood vessel is thinner at this point.

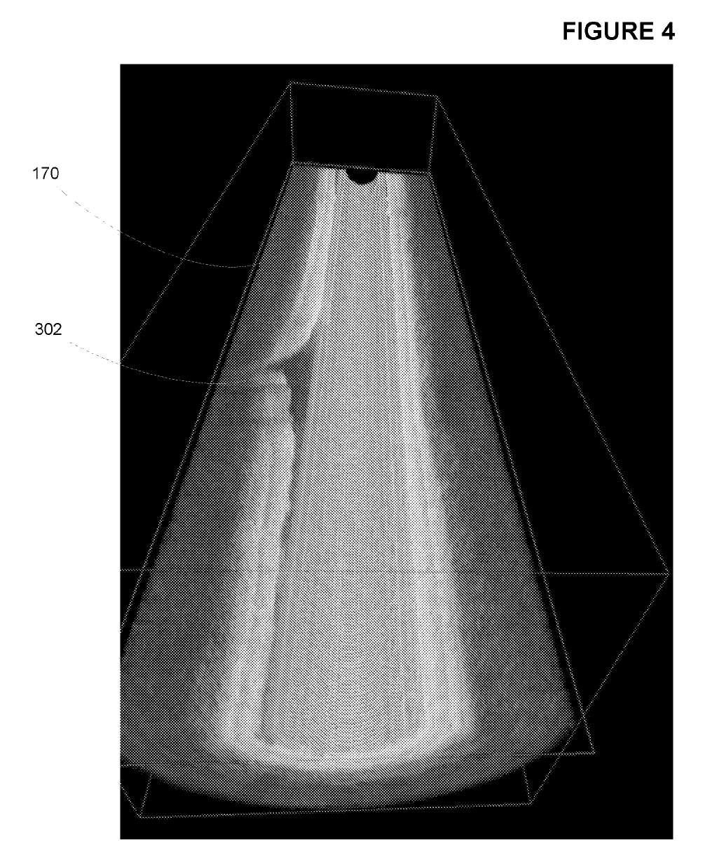

FIG. 4 is a reconstructed three-dimensional cutaway view of blood vessel 170 as captured via an optical tomographic package coupled with the measurement catheter. Endothelial disorder 302 is shown here lengthwise along the axis of blood vessel 170. Embodiments of the invention allow for internal tomographic image capture while the measurement catheter is being moved through the body by associating received light with the position at which the measurement catheter is currently at as determined for example by catheter measurement device 501. Alternatively, flouroscopy or wireless signals may be utilized to determine the position of the measurement catheter to allow for axial construction of the tomographic images along the axis of the artery for example, alternatively, metal ring 108 may be utilized via X-ray imaging techniques to position the measurement catheter.

FIG. 5 is catheter measurement device 501 (for measuring distance, or velocity or any other derivative of distance with respect to time for example such as acceleration, jerk, etc.) configured to measure the distance of the measurement catheter inside a patient. As catheter tube 106 is pushed or pulled through catheter measurement device 501, the distance inside a patient is measured by counting the number of lines along catheter tube 106. In another embodiment of the invention a roller may be utilized to count the number of rotations of a wheel inside catheter measurement device 501 for example to calculate the distance that measurement catheter 100 has traveled into a patient's body. Catheter measurement device 501 is an optional device that is not required for use with embodiments of the measurement catheter and any other method of determining position or velocity of the measurement catheter may be utilized with embodiments of the measurement catheter. Other embodiments of catheter measurement device 501 may be collocated with the insertion point of the catheter and count the number of lines on catheter tube 106. Catheter measurement device 501 may couple with computer system 502 that computes the blood flow velocity and/or constructs the tomographic images.

FIG. 6 is a view of an optical tomographic package having pairs of fiber optic output and input strands pointing radially outward from the package and configured to provide and gather light for optical tomography. In the figure, two embodiments are shown where in the top embodiment the pairs lie side by side radially at optical element 602, while in the bottom embodiment the pairs lie forward and backward at optical element 603 along the axis of measurement catheter 100a and 100b respectively. Shown in the figure are small arrows depicting the direction of photon flow to/from the respective embodiments. Guidewire hole 601 allows for measurement catheters 100a and 100b to move along the guidewire through blood vessels for example (not shown in embodiment 100b for brevity). So long as the fiber pairs are near one another they may be positioned at the same distance along the axis (100a) or forward/behind (100b) or at any other angle between these two alternate configurations. Alternatively, individual lines may be utilized to both transmit and receive light. In single or pair embodiments of the invention, the capture of light by a fiber indicates the angular direction about the measurement catheter lengthwise axis. This allows the measurement catheter to capture optical images without rotating the measurement catheter. By adjusting a reference mirror away from a beam splitter, different depths in the actual arteries may be imaged to provide true three-dimensional tomographic images. Regardless of the optical element embodiment, the fibers that lead to and away from the optical element exist the catheter as fiber bundle 610 that may be utilized for example with an interferometer (see FIG. 7 for example).

One method of manufacturing the optical tomographic package is to assemble a number of fibers of the desired distance (generally long enough to trace the length of associated guidewire and also couple to the OCT/interferometry unit for coupling with computer system 502. For example a length of 3 meters for the fibers allows for insertion of embodiments of the measurement catheter into a body while allowing the light to/from the optical unit. Any other length may be utilized so long as light may be transmitted and obtained from the measurement catheter.

By wrapping a strand of fiber around two drums for example 1.5 meters apart, and then looping the fiber 120 times, flattening the fibers so that they lie side by side and then binding one side of the fibers with an adhesive which is then cut along the axis of the first drum, a 3 meter cable with 120 parallel fibers is thus created. The fibers may then be wrapped around a jig slightly larger than the diameter of a guidewire for example and bound in a medically acceptable plastic. On one end of the 120 lines, the lines may be individually separated and pointed outwardly and bound in that direction. The opposing ends of each fiber may thus be coupled to a electro-optical gates or mechanically positioned for acceptance of a mechanically rotated light source for example. (See FIG. 7). The optical embodiment may be coupled to the thermal embodiment by running wires along the fiber, wherein the optical elements may couple to an optical unit such as an OCT or interferometry unit (which then couples to computer system 502) and the thermal elements may couple with computer system 502 or an analog to digital converter that couples with computer system 502 for example. Although any number of fibers can be utilized, 120 fibers allow for a 3 degree angular resolution of tomographic images to be obtained as will be explained further below in relation to FIG. 7.

FIG. 7 shows views of electro-optical embodiment 710 and a mechanical embodiment 720 of the radial transmitting and receiving portion of the fiber optic elements that direct light to/from optical element 701 (or 602 or 603 for example as per FIG. 6). In addition, on the left side of the figure, the light radiates from and is received by individually radially displaced fibers. The direction of light flow is shown by the arrows wherein the optical element 701 that directs light away from the axis of the catheter is shown as slightly off center in perspective view. The light can be directed at any non-zero angle away from the axis. There is no requirement that the light must be at a 90 degree angle to the axis of the catheter and as long as the light travels to/from the catheter at a non-zero angle, objects at a distance away from the catheter may be imaged. In the first embodiment, at the top right, semiconductor or optoelectronic tree 702 is utilized to switch coherent light into one of the fibers for a desired amount of time. The gates allow for transmitting and receiving light. As shown a binary tree is utilized in one or more embodiments. Other embodiments may utilize gates that switch between more than two paths, and hence binary trees are not required. A gate with 360 possible paths may utilize a single optoelectronic switch and hence not utilize a tree. By sequentially rotating the light around the measurement catheter and receiving the light on each fiber, an optical tomographic image may be created without requiring rotation of the catheter or line running to the catheter, for example with a motor. In order to construct the measurement catheter fiber optics, each line may be redirected from the axial direction of travel along the guidewire, to a non-zero outwardly direction of travel with respect to the catheter axis wherein the light leaves/arrives at optical element 701 of the measurement catheter. (See description above with relation to FIG. 6). In this manner, light can be transmitted and received in each radial direction about the catheter, with the number of fibers determining the radial resolution. By dividing 360 degrees by the number of fibers, the angular resolution may thus be determined. For example 100 fibers that are configured to both transmit and receive would give a 3.6 degree angular resolution. Other embodiments of the invention allow for a conical or faceted mirror having a number of facets equal to the number of fibers wherein the ends of the fibers are not pointed away from the axis of the catheter, but wherein the light bounces off of the end mirror and radiates away from and back to the axis of the catheter.

As shown, reference mirror 711, may be moved closer or away from beam splitter 712 to image in depth away from the catheter's optical element 701. Light source 713 may be any type of light source such as a superluminescent diode, coherent laser or any other type of light source that may be utilized to obtain optical tomographic images. The transmitted light hits beam splitter 712 and travels down each gated fiber coupled to optoelectronic tree 702 (which can simply be a switch depending on the number of desired fibers) where it radiates away from optical element 701. The light hits object such as blood and vessel walls and returns down an associated fiber pair (as per 603 in FIG. 6) or via the same fiber as shown in this figure. When the light passes through beam splitter 712 it combines with light that has passed through beam splitter 712 that traveled to reference mirror 711 and which returns back to the beam splitter and onto imager 714. Imager 714 captures an image of non-interfered light and passes the captured image to computer system 502 for tomographic reconstruction of an image at a particular distance into the vessel and at varying depths as obtained by adjusting reference mirror 711.

Mechanical embodiment 720 is also shown wherein the mechanical movement is distant from the actual measurement catheter. This embodiment moves the light source between the fibers without rotating the measurement catheter itself. In this embodiment, the beam splitter 712 may be located behind motor 721 and use the same interferometric components as used in embodiment 710. By rotating light passing element 723 (for example an opening in disk 722), each of the fibers is provided light that travels to optical element 701 and back and combines with the beam split reference light to be captured by imager 714 as in the case of embodiment 710 above.

By constructing the measurement catheter without regard to sequential placement of fibers, the fiber angular offsets may be determined by firing light into each fiber and determining where about the 360-degree radial area where the light is thus detected. Hence, a mapper 703 may be utilized to convert the signals to occur in sequential order radially by being programmed to map input light to output light path numbers. This is not required however and the individual fiber inputs when received may be thus assigned to their correct angular position programmatically as well.

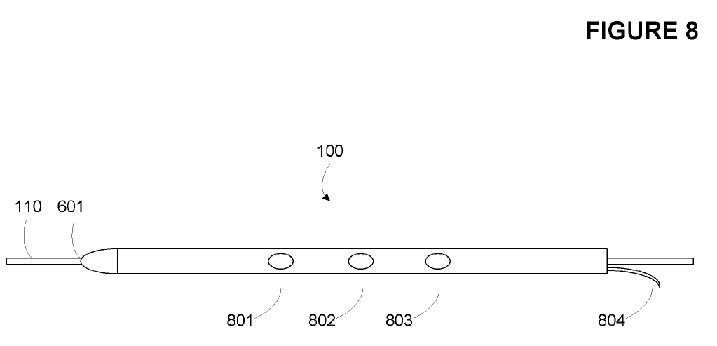

FIG. 8 is a view of a thermal package having a thermal element and thermal sensor configured to measure blood flow velocity. In this embodiment of the invention, there are no fiber pairs coupled to measurement catheter 100c, as this embodiment is directed at blood flow velocity measurement. Thermal changes are introduced at thermal element 801 and sensed at thermal sensors 802 and 803. Thermal element and sensor leads 804 exit the catheter and may be coupled to an analog to digital converter directly or via computer 502 for example.

FIG. 9 is a time display of the thermal sensor after introduction of a thermal change in the blood temperature at the thermal element to determine blood flow velocity. As introduced via thermal element 801 as shown in the bottom most three thermal “humps”, the temperature changes reach thermal sensor 802 slightly offset in time. As the distance between thermal element 801 and thermal sensor 802 is known, the blood flow velocity is thus calculated as Vb=d/T, where Vb is the blood flow velocity and d is the distance between thermal element 801 and thermal sensor 802 and ΔT is the time difference from introduction of a thermal change (for example to blood flowing through a vein), i.e., T1 to the time that the thermal change is observed at thermal sensor 802, i.e., T2. Generally, small changes in temperature are utilized so as not to damage the blood. Any thermistor may be utilized for thermal element 801 and thermal sensor 802 as long as the thermistors do not overheat the blood and are sensitive enough to sense temperature changes that are introduced a distance d away.

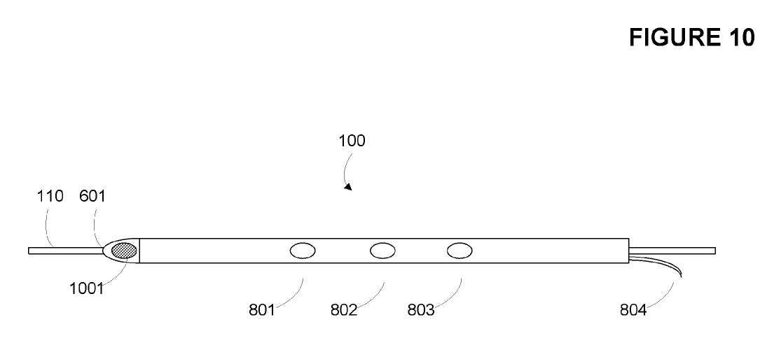

FIG. 10 is a view of the measurement catheter coupled with a tool for use inside of a blood vessel for example. In this example a plaque excision device 1001 is coupled to the end of measurement catheter 100 that is activated to expose and rotate blades to remove plaque from an artery wall for example. The plaque is collected by device 1001 or in another slot near device 1001 depending on the manufacture of the particular excision device so as not to flow into the artery. Example scrapers are made by Fox Hollow® for example under the trademark SILVERHAWK®.

FIG. 11 is a flowchart showing the steps for manufacturing an embodiment of the measurement catheter that includes thermistors for use as a thermal element and thermal sensor(s). In one or more embodiments of the invention, thermistors having 2.1 KOhms at 25 degrees C. and 1.25 KOhms at 39 degrees C. are soldered with non-Lead solder to parallel conducting wire of 38 AWG, for example solid Nickel Bifilar with polyester insulation at 1101 and 1102. The thermistors with wire leads attached are then optionally cleaned in an ultrasonic cleaner for 15 minutes at 1103. The thermistors with wire leads are then aged at 105 degrees C. for 4 days at 1104. Two or more thermistors are then bound in a plastic casing at an offset in distance along the wire at 1205 and cured for 4 hours at 70 degrees C. at 1106. The offset in distance may be measured and utilized for example in blood flow velocity calculations. Alternatively, the distance may be indirectly measured by placing the measurement catheter in a liquid with calibrated flow rate. The distance is then equal to the known velocity V multiplied by the time offset of a thermal event initiated at one thermistor and observed on the other. Any derivations of these steps or specific elements utilized in these steps may be performed so long as the thermistors are bound inside the catheter at an offset from one another. Any type of medical grade plastic can be used to bind the thermistors. Any variance of the materials listed above may be undertaken so long as the thermistors used can assert and obtain thermal events that may be detected by computer system 502 (or an A/D connected to computer system 502 for example).

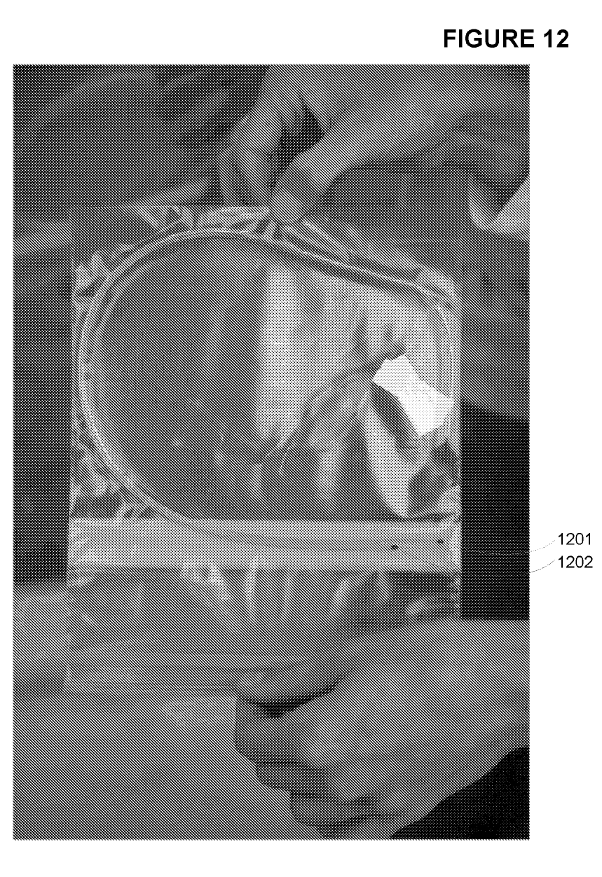

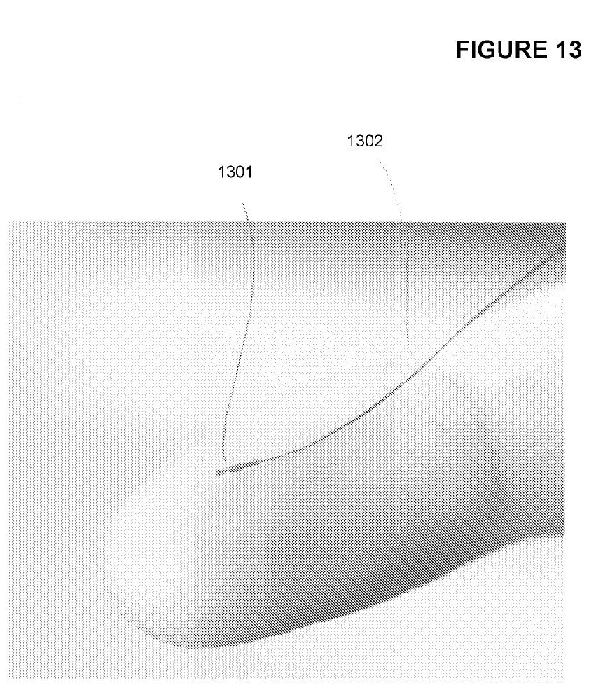

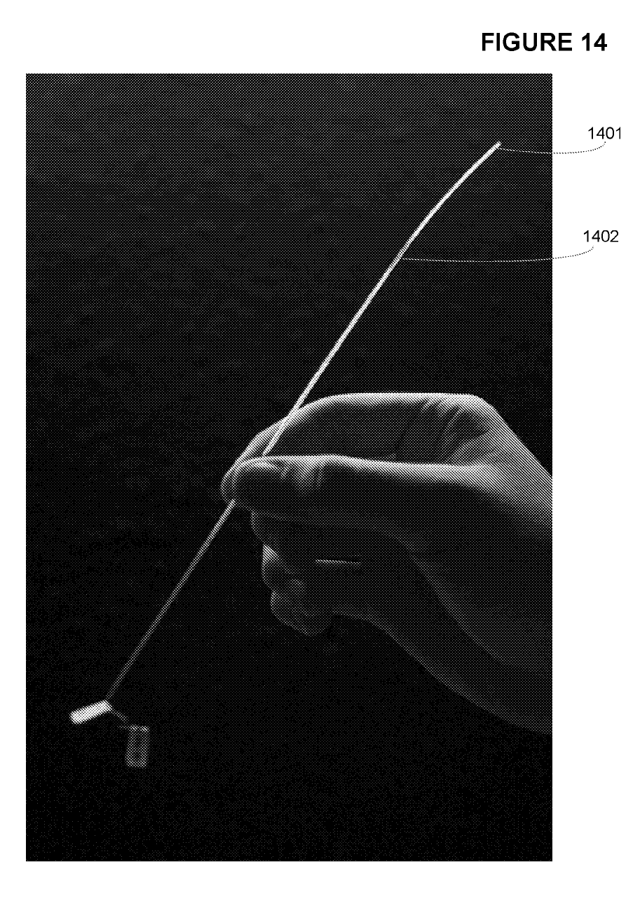

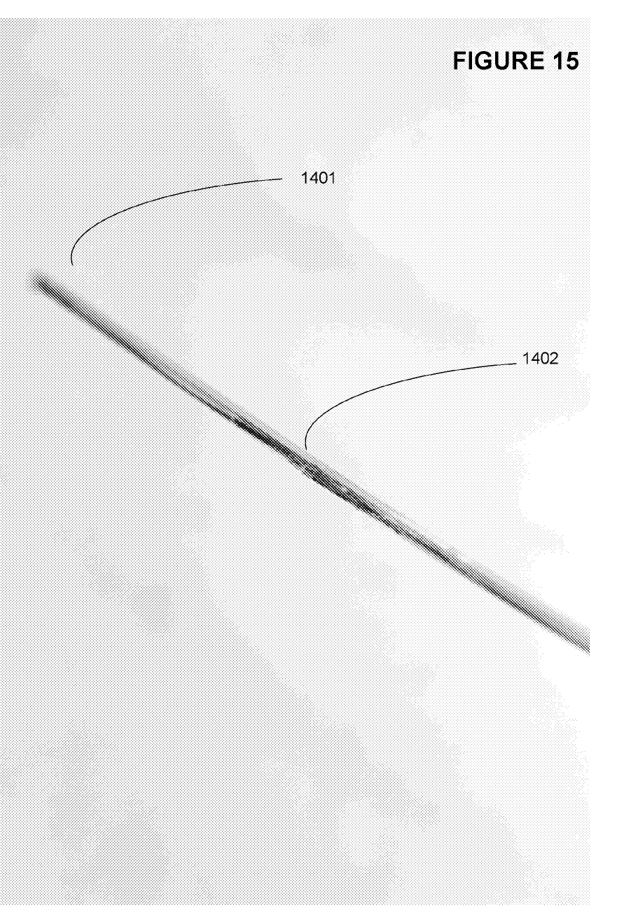

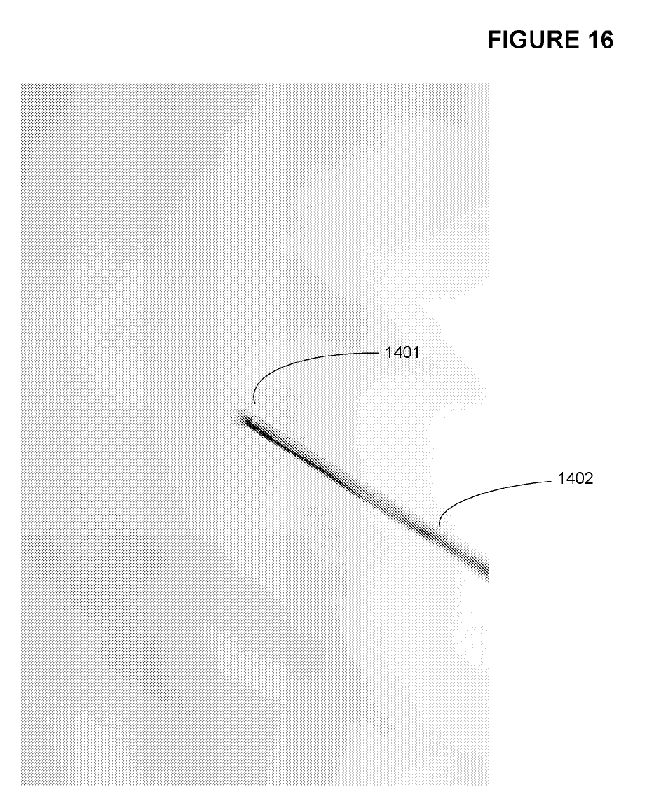

FIG. 12 is a picture of a thermal embodiment of the measurement catheter showing thermal element 1201 and thermal sensor 1202. The thermistors chosen are generally chosen to meet the specific Fr gauge requirements for the desired application. FIG. 13 is a photograph of thermistor 1301 and lead wires 1302 for use in the thermal embodiment for thermal element(s) and thermal sensor(s) as shown on a human finger. Once coupled to wires, the thermistor is ready for coupling to a package or mounting in the measurement catheter directly. FIG. 14 is a picture of a second thermal embodiment of the measurement catheter having thermal element 1401 and thermal sensor 1402. This embodiment is a 5 Fr gauge embodiment manufactured as per the flow diagram of FIG. 12. FIG. 15 is a side view of the measurement catheter shown in FIG. 14 having thermistor 1401 and 1402. FIG. 16 is a close-up picture of the thermal element portion of the thermal embodiment of the measurement catheter of FIG. 15.

While the invention herein disclosed has been described by means of specific embodiments and applications thereof, numerous modifications and variations could be made thereto by those skilled in the art without departing from the scope of the invention set forth in the claims.

What is claimed is:

1. A measurement catheter comprising:
a catheter;
a thermal element coupled with said catheter wherein said thermal element is configured to alter blood temperature as said blood flows through a vessel;
a thermal sensor coupled with said catheter at an offset from said thermal element wherein said thermal sensor is configured to measure said blood temperature and wherein a blood flow velocity is calculated as said offset divided by a time difference from a point in time when said blood temperature is altered at said thermal element to a point in time wherein an altered blood temperature is measured at said thermal sensor;
an optical transmitter coupled with said catheter; and,
an optical sensor coupled with said catheter wherein said optical sensor receives coherent radiation transmitted from said optical transmitter to generate an optical tomographic image of said vessel without rotation of said catheter.

2. The measurement catheter of claim 1 wherein said thermal element is a heating element.

3. The measurement catheter of claim 1 wherein said thermal element is a cooling element.

4. The measurement catheter of claim 1 wherein said thermal sensor comprises a plurality of sensors offset from said thermal element.

5. The measurement catheter of claim 1 wherein said optical transmitter is a broadband light source.

6. The measurement catheter of claim 1 wherein said optical transmitter is a diode.

7. The measurement catheter of claim 1 wherein said optical transmitter is a superluminescent diode.

8. The measurement catheter of claim 1 further comprising an interchangeable coupler configured to mechanically couple a package to said measurement catheter.

9. A measurement catheter comprising:
a catheter;
a thermal element coupled with said catheter wherein said thermal element is configured to alter blood temperature as said blood flows through a vessel; and,
a thermal sensor coupled with said catheter at an offset from said thermal element wherein said thermal sensor is configured to measure said blood temperature and wherein a blood flow velocity is calculated as said offset divided by a time difference from a point in time when said blood temperature is altered at said thermal element to a point in time wherein an altered blood temperature is measured at said thermal sensor.

10. The measurement catheter of claim 9 wherein said thermal element is a heating element.

11. The measurement catheter of claim 9 wherein said thermal element is a cooling element.

12. The measurement catheter of claim 9 wherein said thermal sensor comprises a plurality of sensors offset from said thermal element.

13. The measurement catheter of claim 9 further comprising an interchangeable coupler configured to mechanically couple with said thermal element and said thermal sensor.

14. A measurement catheter comprising:
a catheter;
an optical transmitter coupled with said catheter; and,
an optical sensor coupled with said catheter wherein said optical sensor receives coherent radiation transmitted from said optical transmitter to generate an optical tomographic image of said vessel without rotation of said catheter.

15. The measurement catheter of claim 14 wherein said optical transmitter is a broadband light source.

16. The measurement catheter of claim 14 wherein said optical transmitter is a diode.

17. The measurement catheter of claim 14 wherein said optical transmitter is a superluminescent diode.
